(19) United States
(12) Patent Application Publication
(10) Pub. No.: US 2026/0363868 A1
(43) Pub. Date: Nov. 19, 2026

(54) SUBSCAPULARIS SPARING STEM ADAPTER AND HUMERAL HEAD ADAPTER

(71) Applicant: Zimmer, Inc., Warsaw, IN (US)

(72) Inventor: Michael Mueller, Warsaw, IN (US)

(21) Appl. No.: 19/640,127

(22) Filed: Apr. 6, 2026

Related U.S. Application Data
(60) Provisional application No. 63/806,652, filed on May 15, 2025.

(51) Int. Cl. A61F 2/40

(52) U.S. Cl. CPC A61F 2/4014; A61F 2002/4037

(57) ABSTRACT

A shoulder implant can include a stem member including a humeral head adapter coupling portion extending from a proximal end of the stem member, wherein the humeral head adapter coupling portion includes a tapered female dovetail, and a humeral head adapter configured to couple to the stem member, wherein the humeral head adapter includes a taper connection to couple to a humeral head and a double taper male dovetail configured to mate with the tapered female dovetail of the stem member.

### CLAIM OF PRIORITY

This application claims the benefit of U.S. Provisional Patent Application Ser. No. 63/806,652, filed on May 15, 2025, the benefit of priority of which is claimed hereby, and which is incorporated by reference herein in its entirety.

### TECHNICAL FIELD

The present application relates to an orthopedic system and specifically to a shoulder implant system.

### BACKGROUND

In a healthy shoulder, the proximal humerus is generally ball-shaped, and articulates within a socket formed by the scapula, called the glenoid, to form the shoulder joint. Some implant systems for the replacement of the shoulder joint generally replicate the natural anatomy of the shoulder. Such implant systems can include a humeral component and an articulating head that articulates within the socket of a glenoid component implanted within the glenoid of the scapula.

A typical shoulder replacement operation begins with the detachment of the subscapularis, one of the four rotator cuff muscles responsible for arm mobility and shoulder stability that surrounds the bones of the shoulder. However, after typical shoulder surgery, the patient is required to wear a sling and restrict movement to allow healing of the subscapularis muscle. To ensure the muscle healing, patients must limit strenuous activity and vigorous movement for several weeks after surgery.

In a subscapularis-sparing approach to shoulder arthroplasty the surgeon uses a modified technique to minimize damage to the subscapularis muscle. Since the subscapularis is not detached or only partially detached during surgery, it does not need to be protected or immobilized as long, and this means the patient can more quickly resume normal activities. However, in such an approach, the surgeon does not have much joint space in which to work.

### OVERVIEW

In some embodiments, the present disclosure provides an apparatus and method for a subscapularis sparing shoulder implant. The present disclosure provides a tapered dovetail design that does not require the humeral head to traverse over or navigate around a taper on the humeral stem that is in interference with the joint space. The dovetail design can make humeral head insertion much easier and can prevent the head from potentially compacting the surrounding soft tissue.

In one example, the present disclosure provides a shoulder implant including a stem member including a humeral head adapter coupling portion extending from a proximal end of the stem member, wherein the humeral head adapter coupling portion includes a tapered female dovetail, and a humeral head adapter configured to couple to the stem member, wherein the humeral head adapter includes a taper connection to couple to a humeral head and a double taper male dovetail configured to mate with the tapered female dovetail of the stem member.

In one example the present disclosure provides system for a shoulder implant which can include a humeral stem configured to be implanted in a humerus, a stem adapter configured to couple to the humeral stem and having a humeral head adapter coupling portion extending from a proximal end of the stem adapter, wherein the humeral head adapter coupling portion includes a tapered female dovetail, a humeral head, and a humeral head adapter configured to couple to the stem adapter, wherein the humeral head adapter includes a taper connection to couple to the humeral head and a double taper male dovetail configured to mate with the tapered female dovetail of the stem adapter.

In one example, the present disclosure provides a method of implanting a shoulder implant. The method can include implanting a humeral member into a humerus, the humeral member including a humeral stem and a stem adapter, wherein the stem adapter is coupled to the humeral stem and includes a humeral head adapter coupling portion extending from a proximal end of the stem adapter, wherein the humeral head adapter coupling portion includes a tapered female dovetail, mounting a humeral head to a humeral head adapter, and mounting the humeral head adapter to the stem adapter, wherein the humeral head adapter includes a double taper male dovetail configured to mate with the tapered female dovetail of the stem adapter.

These and other examples and features of the present apparatuses and methods will be set forth in part in the following Detailed Description. This Overview is intended to provide non-limiting examples of the present subject matter—it is not intended to provide an exclusive or exhaustive explanation. The Detailed Description below is included to provide further information about the present apparatus, systems, and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a side view of a shoulder implant, in accordance with one embodiment.

FIG. 2 shows a side view of a stem adapter and a humeral head adapter, in accordance with one embodiment.

FIG. 3 shows a top perspective view of the stem adapter, in accordance with one embodiment.

FIG. 4 shows an end view of the stem adapter, in accordance with one embodiment.

FIG. 5 shows an end view of the humeral head adapter, in accordance with one embodiment.

FIG. 6 shows a side view of the humeral head adapter, in accordance with one embodiment.

FIG. 7 shows a bottom view of the humeral head adapter, in accordance with one embodiment.

FIG. 8 shows a side view of the humeral head adapter placed on the stem adapter in a first orientation, in accordance with one embodiment.

FIG. 9 shows a side view of the humeral head adapter placed on the stem adapter in a second orientation, in accordance with one embodiment.

FIG. 10 shows a side view of the shoulder implant with the humeral head in the first orientation, in accordance with one embodiment.

FIG. 11 shows a view of a shoulder, in accordance with one embodiment.

FIG. 12 shows a view of the shoulder with a humeral stem and stem adapter implanted in a humerus, in accordance with one embodiment.

FIG. 13 shows a view of the shoulder with a humeral head attached to the stem adapter, in accordance with one embodiment.

FIG. 14 shows a method of implanting a shoulder implant, in accordance with one embodiment.

### DETAILED DESCRIPTION

As used herein, the following directional definitions apply. Anterior and posterior mean nearer the front or nearer the rear of the body, respectively, proximal, and distal mean nearer to or further from the root of a structure, respectively, and medial and lateral mean nearer the sagittal plane or further from the sagittal plane, respectively. The sagittal plane is an imaginary vertical plane through the middle of the body that divides the body into right and left halves.

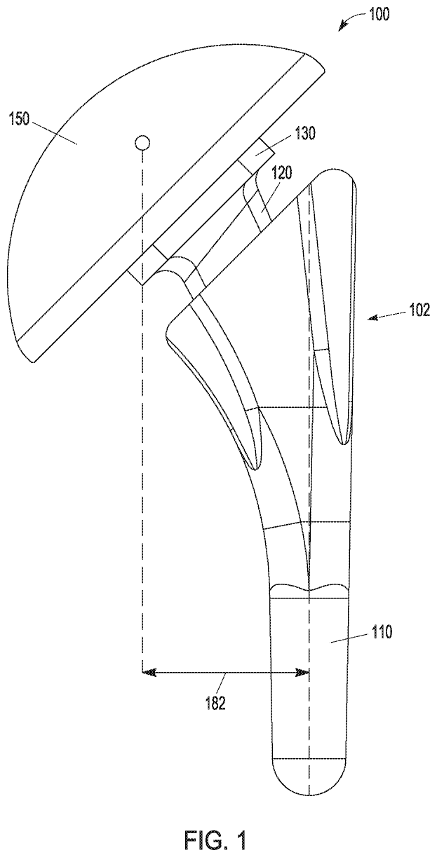

FIG. 1 shows a side view of a shoulder implant 100, in accordance with one embodiment.

The shoulder implant 100 can be used for a subscapularis sparing type implant, as will be further discussed below. The shoulder implant 100 generally includes a stem member 102 and a humeral head 150. The stem member 102 can include a humeral stem 110 configured to be implanted into a humerus and a stem adapter 120 configured to couple to the humeral stem 110 and having a humeral head adapter coupling portion 130 extending from a proximal end of the stem adapter 120. The humeral head 150 can be coupled to the humeral head adapter coupling portion 130.

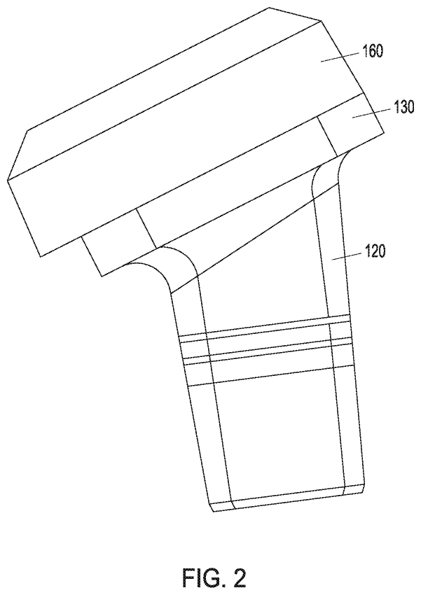

FIG. 2 shows a side view of the stem adapter 120 and an attached humeral head adapter 160, in accordance with one embodiment.

In this example, the stem adapter 120 including the humeral head adapter coupling portion 130. As will be further explained below, the humeral head adapter coupling portion 130 includes a tapered female dovetail.

The humeral head adapter 160 is configured to be coupled to the stem adapter 120 using a dovetail connection. Specifically, in this example, the humeral head adapter 160 can include a double taper male dovetail configured to mate with the tapered female dovetail of the stem adapter 120.

One method of subscapularis sparing shoulder surgery is a superior approach. This surgical approach does not require violating or compromising the subscapularis. However, as noted above, this creates a very small space with soft tissues actively pushing back on retractors. This small space and tight window make it difficult to place humeral heads with a traditional male or female morse taper protruding into the joint space as the taper takes up joint space and the soft tissue may get in the way.

The tapered dovetail approach shown herein does not require the humeral head to traverse over or navigate around a taper that is interference with the joint space. This will make humeral head insertion much easier and will prevent the head from potentially compacting the surrounding soft tissue.

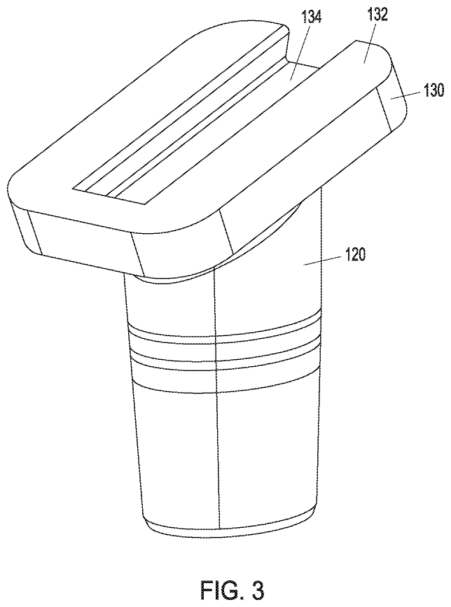

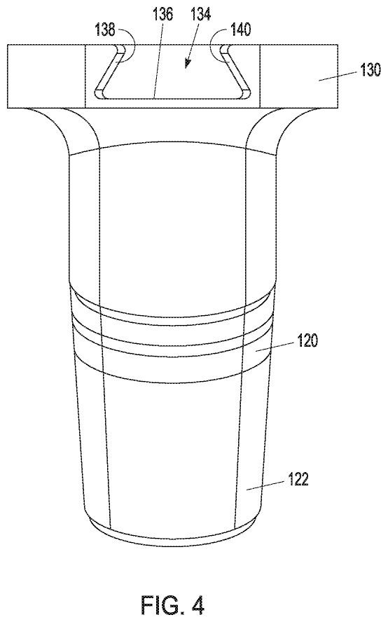

FIG. 3 shows a top perspective view of the stem adapter 120, in accordance with one embodiment, and FIG. 4 shows an end view of the stem adapter 120.

In this example, the stem adapter 120 includes a distal portion 122 defining a stem coupling portion configured to implant into the humeral stem 110 (FIG. 1). For example, the distal portion 122 can include a taper to couple to a female taper of the humeral stem 110. This coupling can be done before implantation into the humerus.

The stem adapter can further include the humeral head adapter coupling portion 130. The humeral head adapter coupling portion 130 can include an upper planar surface 132 with the tapered female dovetail 134 located at a central portion of the planar surface 132. In this example, the humeral head adapter coupling portion 130 can include a generally rectangular shape defining a longitudinal axis and the tapered female dovetail 134 extends along the longitudinal axis.

In this example, the tapered female dovetail 134 extends partially through the humeral head adapter coupling portion 130. In some examples, the female dovetail 134 can extend all the way through the coupling portion 130.

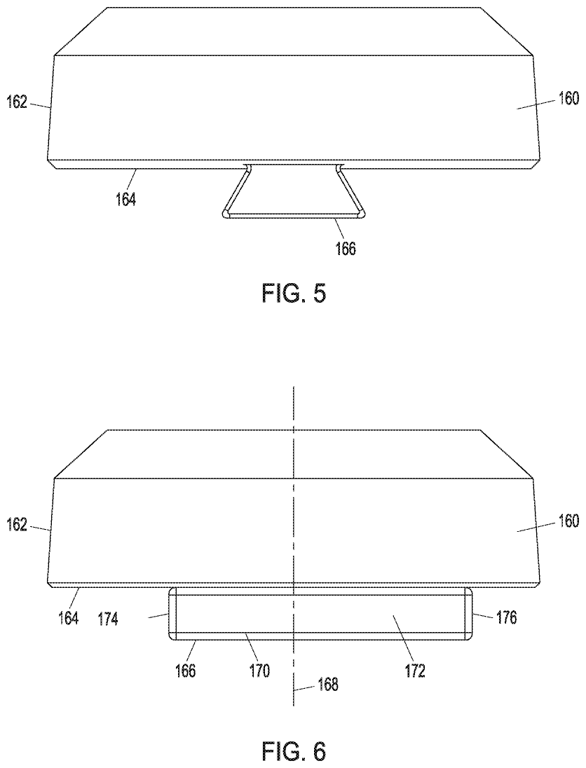

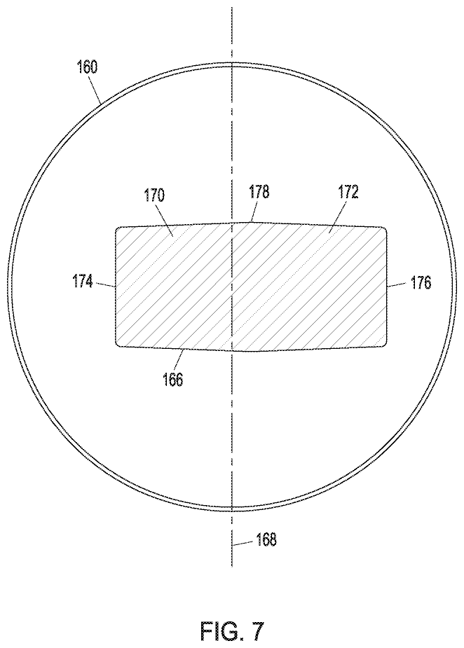

FIG. 5 shows an end view of the humeral head adapter 160, in accordance with one embodiment, FIG. 6 shows a side view of the humeral head adapter 160, and FIG. 7 shows a bottom view of the humeral head adapter 160, in accordance with one embodiment.

The humeral head adapter 160 can include tapered side walls 162 defining a taper to couple to the humeral head 150 (FIG. 1). In an example, this connection can be made prior to implantation using a Morse taper connection with the humeral head 150.

Extending from a bottom surface 164 of the humeral head adapter can be the double taper male dovetail 166 that mates with the female dovetail 134 of the stem adapter 120 (FIGS. 3 and 4). The double taper male dovetail 166 allows the humeral head adapter to be placed on the stem adapter in either of two different orientations.

In this example, the double taper male dovetail 166 defines a longitudinally extending dovetail extending from a first end 174 to a second end 176. The first end 174 includes a first taper portion which is narrower at the first end 174 and wider towards a center 178 of the double taper male dovetail 166. Likewise, the second end 176 includes a second taper portion, which is opposite of the first taper portion, and which is narrower at the second end 176 and wider towards the center 178. Accordingly, the width of the double taper male dovetail goes from being relatively narrow at the first end 174 then widens to the center 178 then narrows again at the second end 176. Thus, the double taper male dovetail 166 includes a first dovetail section extending from first end 174 defining a first taper and an adjacent, second dovetail section extending from the second end 176 defining a second taper having an opposite orientation relative to the first taper.

In one embodiment, the double taper male dovetail 166 is longitudinally offset from a center line 168 of the humeral head adapter 160. The offset is such that the widest part at the center 178 of the double taper male dovetail 166 is offset from the center line 168.

This offset of the double taper male dovetail 166 offers two offset options when installing the humeral head adapter 160 to the stem adapter 120. This allows the humeral head adapter 160 to be placed in two different orientations allowing for min/max offset.

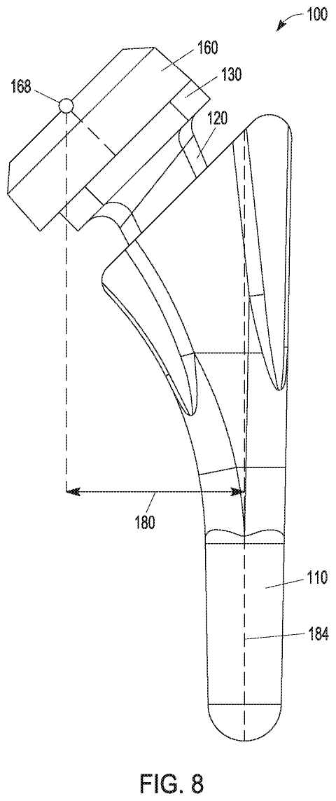

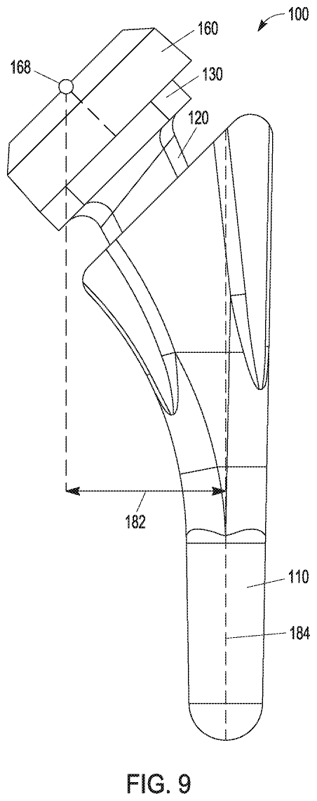

For example, FIG. 8 shows a side view of the humeral head adapter 160 placed on the stem adapter 120 in a first orientation, and FIG. 9 shows a side view of the humeral head adapter 160 placed on the stem adapter 120 in a second orientation, in accordance with one embodiment.

Referring also to FIGS. 5-7, the offset position of the double taper male dovetail 166 is configured such that the humeral head adapter 160 can be positioned on the humeral head adapter coupling portion in a first orientation (FIG. 8) having a maximum offset 180 from a centerline 184 of the stem 110 and in a second opposite orientation (FIG. 9) having a minimum offset 182 from the centerline 184 of the humeral stem 110. By using the offset double taper male dovetail 166 the humeral head adapter 160 can be positioned on the stem adapter 120 in two different orientations having two different offsets.

In one example, in the first orientation, a center point 168 of the humeral head adapter 160 is distanced further laterally from the humeral stem 110 than the center point 168 in the second orientation. For example, there can be a 2 mm difference in the offsets. In some embodiments the offset can be configured to provide a 1 mm offset. In other embodiments, the offset can be 1.5 mm, or 3 mm or higher.

In this example, the humeral head adapter coupling portion 130 defines an upper surface area which is smaller than a surface area defined by a lower distal surface of the humeral head adapter 160 such that at least one or more sides of the humeral head adapter 160 extend over outer edges of the humeral head adapter coupling portion 130

Thus, the humeral head adapter coupling portion 130 and the humeral head adapter 160 can be dimensioned such that in the first orientation (FIG. 8) a front portion of the humeral head adapter 160 overhangs the humeral head adapter coupling portion 130 and in the second orientation (FIG. 9) a back portion of the humeral head adapter 160 overhangs the humeral head adapter coupling portion 130.

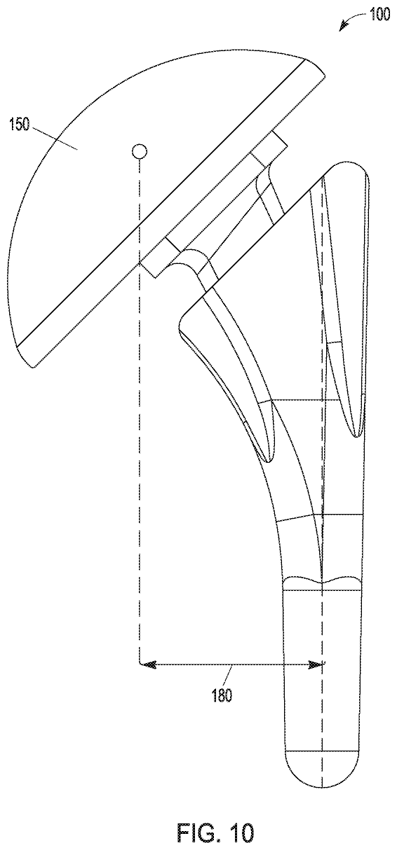

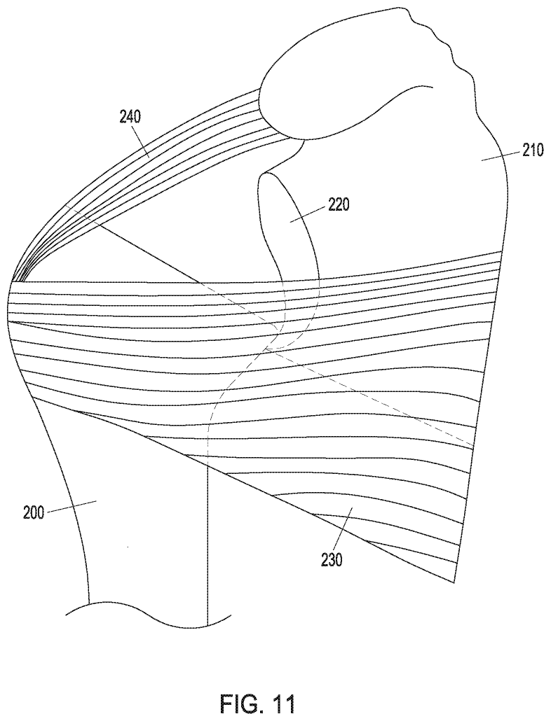

FIG. 10 shows a side view of the shoulder implant 100 with the humeral head 150 in the first orientation with the offset 180, in accordance with one embodiment. In contrast, the shoulder implant 100 of FIG. 1 shows the shoulder implant 100 with the humeral head 150 in the second orientation with the offset 182. Again, the offset double taper male dovetail 166 allows for the user to install the humeral head 150 in the most desired orientation for a proper fit.

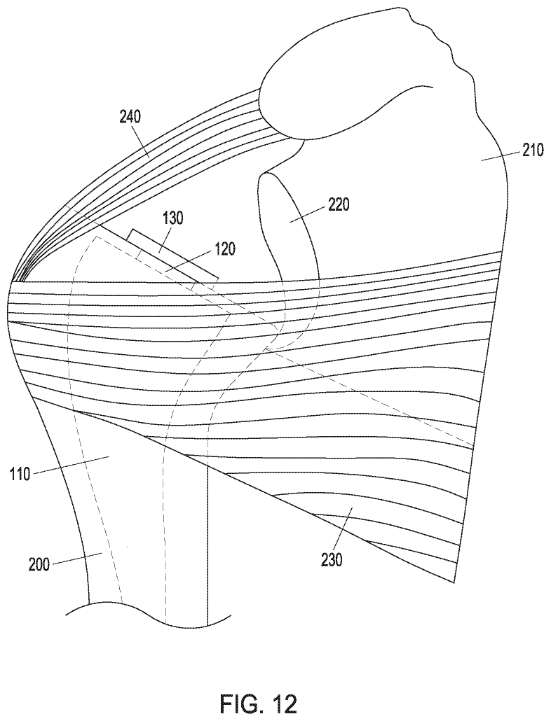

FIGS. 11-13 show an environment for use of the implant system discussed herein.

FIG. 11 shows a view of a shoulder, in accordance with one embodiment.

Generally, the bones of the shoulder joint include a humerus 200, a proximal end of which is shown resected in preparation for an implant, and a scapula 210, which includes a glenoid cavity 220 that forms the socket of the ball-and-socket joint. Also shown are the subscapularis muscle 230 and the supraspinatus muscle 240. Generally, the subscapularis muscle 230 is attached to the lesser tubercle of the humerus 200, and the supraspinatus muscle 240 is attached to the greater tubercle of the humerus 200. The subscapularis muscle 230 and supraspinatus muscle 240 form two of the four muscles forming the rotator cuff. As shown in FIG. 11, a generally triangular space is formed between the tendons of the subscapularis muscle 230 and the tendons of the supraspinatus muscle 240. This triangular space is known as the rotator cuff interval, which provides a space through which instruments may be inserted for a minimally invasive shoulder arthroplasty procedure.

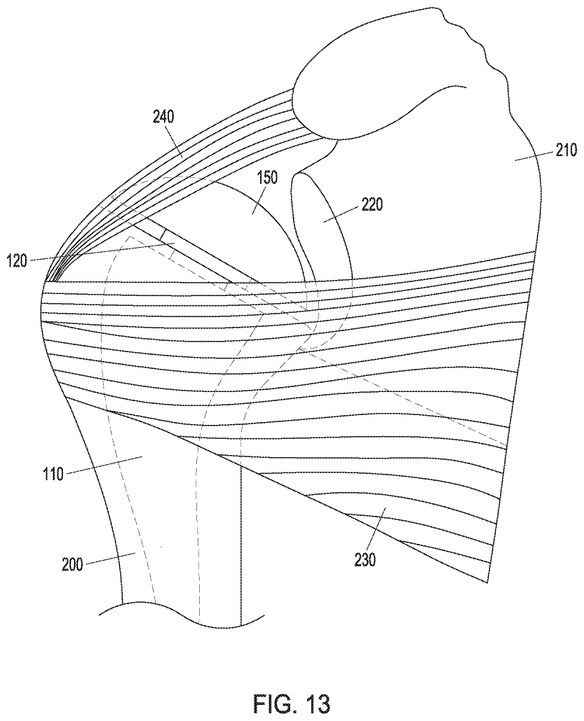

FIG. 12 shows a view of the shoulder with the humeral stem 110 and the stem adapter 120 implanted in the humerus 200, in accordance with one embodiment, and FIG. 13 shows a view of the shoulder with the humeral head 150 attached to the stem adapter 120, in accordance with one embodiment.

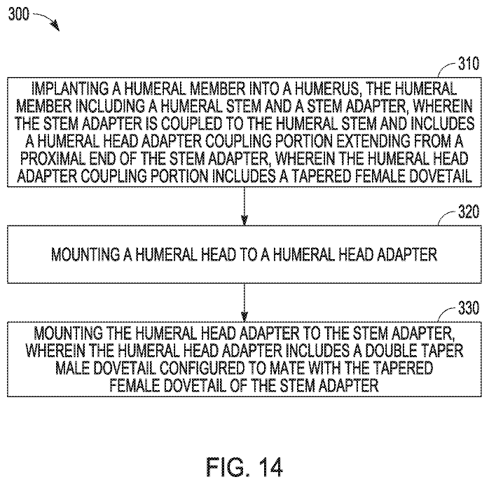

FIG. 14 shows a method (300) of implanting a shoulder implant, in accordance with one embodiment.

Referring to the above FIGS. 1-13, the method (300) can include implanting a humeral member into a humerus, the humeral member including a humeral stem and a stem adaptor, wherein the stem adapter is coupled to the humeral stem and includes a humeral head adapter coupling portion extending from a proximal end of the stem adapter, wherein the humeral head adapter coupling portion includes a tapered female dovetail (310). The method can further include mounting a humeral head to a humeral head adapter (320). This can be done prior to implantation using a Morse taper connection with the humeral head. The method can further include mounting the humeral head adapter to the stem adapter, wherein the humeral head adapter includes a double taper male dovetail configured to mate with the tapered female dovetail of the stem adapter (330).

In various examples, and as discussed above, the method can include the double taper male dovetail defining a dovetail extending from a first end to a second end, wherein the first end includes a first taper portion which is narrower at the first end and wider towards a center of the double taper male dovetail, and the second end includes a second taper portion, which is opposite of the first taper portion, and which is narrower at the second end and wider towards the center.

In one example, the double taper male dovetail is offset from a center point of the humeral head adapter such that the humeral head adapter can be positioned on the humeral head adapter coupling portion in a first orientation and a second opposite orientation. In the first orientation a center point of the humeral head adapter is distanced further laterally from the humeral stem than the center point in the second orientation.

In summary, the present shoulder system and method can be utilized for a subscapularis sparing type implant, for example using a superior approach. In a subscapularis sparing technique, the user has only a small space to work in. This small space and tight window make it difficult to place humeral heads with a traditional male or female morse taper protruding into the joint space as the taper takes up joint space and the soft tissue may get in the way.

The tapered dovetail approach discussed herein does not require the humeral head to traverse over or navigate around a taper on the stem that is in interference with the joint space. This can make humeral head insertion much easier and can prevent the head from potentially compacting the surrounding soft tissue.

Moreover, using an offset double taper male dovetail offers two offset options. (e.g., min/max offset). Providing an offset dovetail connection allows the surgeon to have different options for positioning of the humeral head 150 at either one of a min/max offset.

While the present system can be applicable during a subscapularis sparing shoulder procedure, certain aspects of the present system can also be applicable to a traditional shoulder procedure.

### Additional Notes and Examples

To further illustrate devices, systems and methods disclosed herein, the following non-limiting examples are provided:

Example 1 includes a shoulder implant including a stem member including a humeral head adapter coupling portion extending from a proximal end of the stem member, wherein the humeral head adapter coupling portion includes a tapered female dovetail; and a humeral head adapter configured to couple to the stem member, wherein the humeral head adapter includes a taper connection to couple to a humeral head and a double taper male dovetail configured to mate with the tapered female dovetail of the stem member.

Example 2 is the implant of Example 1, wherein optionally the stem member includes a stem adapter configured to implant into a humeral stem.

Example 3 is the implant of any of Examples 1-2, wherein optionally the double taper male dovetail defines a dovetail extending from a first end to a second end, wherein the first end includes a first taper portion which is narrower at the first end and wider towards a center of the double taper male dovetail, and the second end includes a second taper portion, which is opposite of the first taper portion, and which is narrower at the second end and wider towards the center.

Example 4 is the implant of any of Example 1-3, wherein optionally the double taper male dovetail is offset from a center point of the humeral head adapter.

Example 5 is the implant of any of Examples 1-4, wherein optionally the offset position of the double taper male dovetail is configured such that the humeral head adapter can be positioned on the humeral head adapter coupling portion in a first orientation and a second opposite orientation.

Example 6 is the implant of any of Examples 1-5, wherein optionally in the first orientation a front portion of the humeral head adapter overhangs the humeral head adapter coupling portion and in the second orientation a back portion of the humeral head adapter overhangs the humeral head adapter coupling portion.

Example 7 is the implant of any of Examples 1-6, wherein optionally in the first orientation a center point of the humeral head adapter is distanced further laterally from a humeral stem than the center point in the second orientation.

Example 8 is the implant of any of Examples 1-7, wherein optionally the humeral head adapter coupling portion includes a planar surface with the tapered female dovetail located at a central portion of the planar surface.

Example 9 is the implant of any of Examples 1-8, wherein optionally the tapered female dovetail extends partially through the humeral head adapter coupling portion.

Example 10 is the implant of any of Example 1-9, wherein optionally the tapered female dovetail extends partially through the humeral head adapter coupling portion.

Example 11 includes a system for a shoulder implant including a humeral stem configured to be implanted in a humerus; a stem adapter configured to couple to the humeral stem and having a humeral head adapter coupling portion extending from a proximal end of the stem adapter, wherein the humeral head adapter coupling portion includes a tapered female dovetail; a humeral head; and a humeral head adapter configured to couple to the stem adapter, wherein the humeral head adapter includes a taper connection to couple to the humeral head and a double taper male dovetail configured to mate with the tapered female dovetail of the stem adapter.

Example 12 is the system of Examples 11, wherein optionally the double taper male dovetail defines a dovetail extending from a first end to a second end, wherein the first end includes a first taper portion which is narrower at the first end and wider towards a center of the double taper male dovetail, and the second end includes a second taper portion, which is opposite of the first taper portion, and which is narrower at the second end and wider towards the center.

Example 13 is the system of any of Examples 11-12, wherein optionally the double taper male dovetail is offset from a center point of the humeral head adapter.

Example 14 is the system of any of Examples 11-13, wherein optionally the offset position of the double taper male dovetail is configured such that the humeral head adapter can be positioned on the humeral head adapter coupling portion in a first orientation and a second opposite orientation.

Example 15 is the system of any of Examples 11-14, wherein optionally in the first orientation a center point of the humeral head adapter is distanced further laterally from the humeral stem than the center point in the second orientation.

Example 16 is the system of any of Examples 11-15, wherein optionally the humeral head adapter coupling portion includes a rectangular shape defining a longitudinal axis and the tapered female dovetail extends along the longitudinal axis.

Example 17 is a method of implanting a shoulder implant including implanting a humeral member into a humerus, the humeral member including a humeral stem and a stem adapter, wherein the stem adapter is coupled to the humeral stem and includes a humeral head adapter coupling portion extending from a proximal end of the stem adapter, wherein the humeral head adapter coupling portion includes a tapered female dovetail; mounting a humeral head to a humeral head adapter; and mounting the humeral head adapter to the stem adapter, wherein the humeral head adapter includes a double taper male dovetail configured to mate with the tapered female dovetail of the stem adapter.

Example 18 is the method of Example 17, wherein optionally the double taper male dovetail defines a dovetail extending from a first end to a second end, wherein the first end includes a first taper portion which is narrower at the first end and wider towards a center of the double taper male dovetail, and the second end includes a second taper portion, which is opposite of the first taper portion, and which is narrower at the second end and wider towards the center.

Example 19 is the method of any of Examples 17-18, wherein optionally wherein the double taper male dovetail is offset from a center point of the humeral head adapter such that the humeral head adapter can be positioned on the humeral head adapter coupling portion in a first orientation and a second opposite orientation.

Example 20 is the method of any of Examples 17-19, wherein optionally in the first orientation a center point of the humeral head adapter is distanced further laterally from the humeral stem than the center point in the second orientation.

In Example 21, the apparatuses, systems, or methods of any one or any combination of Examples 1-20 can optionally be configured such that all elements or combinations of elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive.

For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A shoulder implant comprising:
a stem member including a humeral head adapter coupling portion extending from a proximal end of the stem member, wherein the humeral head adapter coupling portion includes a tapered female dovetail; and
a humeral head adapter configured to couple to the stem member, wherein the humeral head adapter includes a taper connection to couple to a humeral head and a double taper male dovetail configured to mate with the tapered female dovetail of the stem member.

2. The implant of claim 1, wherein the stem member includes a stem adapter configured to implant into a humeral stem.

3. The implant of claim 1, wherein the double taper male dovetail defines a dovetail extending from a first end to a second end, wherein the first end includes a first taper portion which is narrower at the first end and wider towards a center of the double taper male dovetail, and the second end includes a second taper portion, which is opposite of the first taper portion, and which is narrower at the second end and wider towards the center.

4. The implant of claim 3, wherein the double taper male dovetail is offset from a center point of the humeral head adapter.

5. The implant of claim 4, wherein the offset position of the double taper male dovetail is configured such that the humeral head adapter can be positioned on the humeral head adapter coupling portion in a first orientation and a second opposite orientation.

6. The implant of claim 5, wherein in the first orientation a front portion of the humeral head adapter overhangs the humeral head adapter coupling portion and in the second orientation a back portion of the humeral head adapter overhangs the humeral head adapter coupling portion.

7. The implant of claim 6, wherein in the first orientation a center point of the humeral head adapter is distanced further laterally from a humeral stem than the center point in the second orientation.

8. The implant of claim 1, wherein the humeral head adapter coupling portion includes a planar surface with the tapered female dovetail located at a central portion of the planar surface.

9. The implant of claim 8, wherein the humeral head adapter coupling portion includes a rectangular shape defining a longitudinal axis and the tapered female dovetail extends along the longitudinal axis.

10. The implant of claim 9, wherein the tapered female dovetail extends partially through the humeral head adapter coupling portion.

11. A system for a shoulder implant comprising:
a humeral stem configured to be implanted in a humerus;
a stem adapter configured to couple to the humeral stem and having a humeral head adapter coupling portion extending from a proximal end of the stem adapter, wherein the humeral head adapter coupling portion includes a tapered female dovetail;
a humeral head; and
a humeral head adapter configured to couple to the stem adapter, wherein the humeral head adapter includes a taper connection to couple to the humeral head and a double taper male dovetail configured to mate with the tapered female dovetail of the stem adapter.

12. The system of claim 11, wherein the double taper male dovetail defines a dovetail extending from a first end to a second end, wherein the first end includes a first taper portion which is narrower at the first end and wider towards a center of the double taper male dovetail, and the second end includes a second taper portion, which is opposite of the first taper portion, and which is narrower at the second end and wider towards the center.

13. The system of claim 12, wherein the double taper male dovetail is offset from a center point of the humeral head adapter.

14. The system of claim 13, wherein the offset position of the double taper male dovetail is configured such that the humeral head adapter can be positioned on the humeral head adapter coupling portion in a first orientation and a second opposite orientation.

15. The system of claim 14, wherein in the first orientation a center point of the humeral head adapter is distanced further laterally from the humeral stem than the center point in the second orientation.

16. The system of claim 11, wherein the humeral head adapter coupling portion includes a rectangular shape defining a longitudinal axis and the tapered female dovetail extends along the longitudinal axis.

17. A method of implanting a shoulder implant, comprising:
implanting a humeral member into a humerus, the humeral member including a humeral stem and a stem adapter, wherein the stem adapter is coupled to the humeral stem and includes a humeral head adapter coupling portion extending from a proximal end of the stem adapter, wherein the humeral head adapter coupling portion includes a tapered female dovetail;
mounting a humeral head to a humeral head adapter; and
mounting the humeral head adapter to the stem adapter, wherein the humeral head adapter includes a double taper male dovetail configured to mate with the tapered female dovetail of the stem adapter.

18. The method of claim 17, wherein the double taper male dovetail defines a dovetail extending from a first end to a second end, wherein the first end includes a first taper portion which is narrower at the first end and wider towards a center of the double taper male dovetail, and the second end includes a second taper portion, which is opposite of the first taper portion, and which is narrower at the second end and wider towards the center.

19. The method of claim 18, wherein the double taper male dovetail is offset from a center point of the humeral head adapter such that the humeral head adapter can be positioned on the humeral head adapter coupling portion in a first orientation and a second opposite orientation.

20. The method of claim 19, wherein in the first orientation a center point of the humeral head adapter is distanced further laterally from the humeral stem than the center point in the second orientation.
